(19) United States
(12) Patent Application Publication
(10) Pub. No.: US 2010/0272825 A1
(43) Pub. Date: Oct. 28, 2010

(54) METHOD OF DISINFECTING CARCASSES

(75) Inventors: Michael F. Coughlin, Cincinnati, OH (US); Michael Hanschke, Mason, OH (US)

Correspondence Address: Diversey, Inc., 8310 16TH STREET, M/S 509, PO BOX 902, STURTEVANT, WI, 53177-0902, US

(73) Assignee: JOHNSONDIVERSEY, INC., Sturtevant, WI (US)

(21) Appl. No.: 12/517,850

(22) PCT Filed: Nov. 20, 2007
(86) PCT No.: PCT/US07/85211

§ 371 Date: Sep. 24, 2009

Related U.S. Application Data
(60) Provisional application No. 60/869,168, filed on Dec. 8, 2006.

(51) Int. Cl. A61L 2/18; A61K 33/00

(52) U.S. Cl. 424/600; 426/332

(57) ABSTRACT

Animal carcasses are disinfected by applying to the surface of the animal carcass an acidified solution of chlorine dioxide. The chlorine dioxide solution should have a pH of about 2-3 and a concentration of 1-100 ppm. The contact time for the solution onto the carcass surface should be from about 5 seconds to about 60 seconds.

### BACKGROUND OF THE INVENTION

Various methods are employed to disinfect animal carcasses prior to distribution and subsequent human consumption. Chlorine dioxide in solution is frequently used as a disinfectant. Unlike chlorine, which is very pH dependent, chlorine dioxide is known to be effective over a wide pH range, generally from 4 to 9. Chlorine dioxide can be prepared either by adding an acid to sodium chlorite or sodium chlorate when used with a reducing agent. In addition, chlorine dioxide can be generated by combining sodium chloride with sodium hypochlorite and an acid. Chlorine dioxide can also be generated by an ion exchange resin.

These acidic solutions are highly concentrated with up to 20,000 ppm chlorine dioxide. When applied to disinfect an animal carcass, the solutions are diluted to 1 ppm up to 100 ppm, which increases the pH of the solution due to its dilution with water.

### SUMMARY OF THE INVENTION

The present invention is premised on the realization that a dilute solution of chlorine dioxide more effectively kills bacteria on an animal carcass in a short contact time at a pH of about 2-3. More particularly, according to the present invention, animal carcasses such as poultry carcasses are sprayed with a solution of chlorine dioxide at a pH of 2-3 with a contact time of less than 1 minute to destroy bacteria on the surface of the animal carcass.

The objects and advantages of the present invention will be further appreciated in light of the following detailed description.

### DETAILED DESCRIPTION

According to the present invention, animal carcasses are sprayed with an acidic solution of chlorine dioxide to destroy bacteria on the surface of the carcass.

Any method of producing a solution of chlorine dioxide is suitable for use in the present invention. This can be prepared using, for example, the methods disclosed in U.S. published application 2002/0122854 A1, entitled “Acidified Metal Chlorite Solution for Disinfection of Beef, Pork and Poultry”, or using an ion exchange column as disclosed in U.S. published application 2003/0064018 A1, entitled “Method for Making Chlorous Acid and Chlorine Dioxide”, the disclosures of which are hereby incorporated by reference.

The produced chlorine dioxide solution may have up to about 1000 ppm ClO₂. This concentrated solution is diluted with an acidic solution to achieve an end use solution with about 1 to about 100 ppm ClO₂. Preferably, the concentration is 25-50 ppm ClO₂. The acidic solution should have a pH effective to establish an end use solution having a pH of from about 2 to about 3, with about 2.5 being preferred. This can be accomplished by using a solution of any GRAS listed acid, including organic acids such as citric acid, acetic acid, and the like, as well as inorganic acids such as hydrochloric acid and sulfuric acid, as well as sodium bisulfate and acidification via ion exchange. The preferred acid for use in the present invention is sodium bisulfate.

Once the concentrated chlorine dioxide solution is diluted with the acid solution to establish the desired concentration of ClO₂ and pH, it is immediately applied onto the surface of an animal carcass, such as a poultry carcass. Any typical application method such as immersion or spraying can be used. The preferred method of applying the disinfectant solution is spraying the solution onto the carcass as it passes through a spray booth. Any spray booth typically used for carcass disinfection can be used. Generally, the contact time for the disinfectant solution on the carcasses is anywhere from about 10 seconds to 1 minute, preferably 5-10 seconds. This is accomplished at ambient temperature. The carcass is then ready for packaging and shipment. The used solution is typically discarded but could be recirculated, especially if purified by filtration.

The invention will be further appreciated in light of the following detailed examples. In these examples, a chlorine dioxide solution was obtained by forming chlorous acid from an ion exchange resin, running this through a platinum catalyst to form chlorine dioxide. The chlorine dioxide obtained from the ion exchange resin catalyst combination was generally at about 650-750 ppm at a pH of about 2, which was then diluted with an acid solution of sodium bisulfate to reach the desired use concentration as set forth in the examples.

### EXAMPLE 1

### Results of Sanitizing Effectiveness

| | |
|---|---|
| Test Method: | A modification of the Germicidal and |
| | Detergent Sanitizing Action of Disin- |
| | fectants, A.O.A.C., 17^{th} Edition, 2000. |
| Test Solutions and | Chlorine Dioxide, 1 ppm |
| Concentration: | |
| pH: | All solutions prepared at pH 2.0, 2.5, |
| | 3.0, 3.5, and 4.0 |
| Test Organisms: | Salmonella typhi, ATCC # 6539 |
| | Initial numbers = 116,000,000 org/ml |
| Contact Time: | 10 seconds 25° C. |
| Recovery Medium | Tryptone Glucose Extract Agar with Sodium |
| | thiosulfate |
| Neutralized Medium: | Difco Neutralizing Buffer |
| Incubation: | 72 hours at 35° C. |

| | 1 ppm Chlorine Dioxide | | Acid (no ClO₂) | |
|---|---|---|---|---|
| pH | Survivors | % Reduction | Survivors | % Reduction |
| 2.0 | 1,040,000 | 99.103 | 23,000,000 | 80.172 |
| 2.5 | 920,000 | 99.207 | 69,000,000 | 40.517 |
| 3.0 | >2,500,000 | <97.845 | 37,400,000 | 67.759 |
| 3.5 | >2,500,000 | <97.845 | 34,500,000 | 70.259 |
| 4.0 | >2,500,000 | <97.845 | 28,800,000 | 75.172 |

As shown by Example 1, the chlorine dioxide solution in combination with the acid is a much more effective sanitizer than the acid itself.

### EXAMPLE 2

| | |
|---|---|
| Test Method: | AOAC Chlorine (Available) in Disinfectants, |
| | AOAC Official Methods of analysis, AOAC, |
| | 17^{th} Edition, 2000, 6.3.01, Ch. 6. |
| Subculture Medium: | Thioglycollate Broth |
| Incubation: | 37° C. for 48 hours |
| Product Dilution: | Tested as supplied |
| Organism: | Salmonella typhi, ATCC # 6359 |

| | Subculture Series | | | | | | | | | |
|---|---|---|---|---|---|---|---|---|---|---|
| Test Solution | 1 | 2 | 3 | 4 | 5 | 6 | 7 | 8 | 9 | 10 |
| NaOCl | | | | | | | | | | |
| 200 ppm | − | − | − | − | − | − | − | + | + | + |
| 100 ppm | − | − | − | − | + | + | + | + | + | + |
| 50 ppm | − | + | + | + | + | | | | | |
| ClO₂ | | | | | | | | | | |
| 12 ppm | − | − | + | + | + | + | + | + | + | + |
| pH = 3.82 | | | | | | | | | | |
| 12 ppm | − | − | − | − | + | + | + | + | + | + |
| pH = 2.50 | | | | | | | | | | |
| 15 ppm | − | − | − | − | − | + | + | + | + | + |
| pH = 3.75 | | | | | | | | | | |
| 15 ppm | − | − | − | − | − | − | − | − | + | + |
| pH = 2.50 | | | | | | | | | | |
| Control pH 2.5 | + | + | + | + | + | + | + | + | + | + |

+ = growth
− = no growth

Conclusion: 15 ppm of ClO₂ at pH 2.5 showed germicidal efficacy=200 ppm of sodium hypochlorite while 15 ppm of ClO₂ at pH 3.75 showed efficacy at to >100 ppm and <200 ppm of sodium hypochlorite.

As demonstrated by these Examples and by the general description, the present invention increases the efficacy of chlorine dioxide used as a sanitizer for treating the surface of animal carcasses. This is achieved at relatively low concentrations of chlorine dioxide, over relatively short periods of time, and without any significant increases in costs. Thus, the present invention reduces the risk of bacterial infection from consuming meats, and particularly poultry. Further, at these concentrations, no discoloration of the product was observed.

This has been a description of the present invention along with the preferred method of practicing the present invention. However, the invention itself should only be defined by the appended claims.

What is claimed is:

1. A method of disinfecting an animal carcass comprising applying to said animal carcass a solution of chlorine dioxide having a pH of 2-3, said chlorine dioxide solution having a concentration of chlorine dioxide of about 1:100 ppm.

2. The method claimed in claim 1 comprising spraying said solution of chlorine dioxide onto said animal carcass.

3. The method claimed in claim 2 wherein said animal carcass is contacted with said disinfectant solution for a period of 5-10 seconds.

4. The method claimed in claim 1 wherein a concentrated solution of chlorine dioxide is blended with an acidic solution to establish a desired use concentration wherein said acidic solution is a solution of sodium bisulfate.

5. The method claimed in claim 1 wherein the chlorine dioxide solution has a concentration of chlorine dioxide of about 1 ppm to about 35 ppm.

6. The method claimed in claim 5 wherein the pH of the solution of chlorine dioxide is about 2.5 ppm.

7. The method claimed in claim 1 wherein the pH of the solution of chlorine dioxide is about 2.5 ppm.

8. The method claimed in claim 1 wherein a concentrated solution of chlorine dioxide is blended with an acidic solution to establish a desired use concentration wherein said acidic solution is a solution of GRAS listed acids.

9. The method claimed in claim 1 wherein said animal carcass is contacted with said disinfectant solution for a period of up to one minute.

10. A disinfectant solution for use in disinfecting animal carcasses, the disinfectant solution comprising:
chlorine dioxide; and
an acidic solution having a pH effective to establish an end use disinfectant solution having a pH of from about 2 to about 3;
wherein the end use disinfectant solution has a chlorine dioxide concentration of about 1 to about 100 ppm.

11. The disinfectant solution of claim 10 wherein the end use disinfectant solution has a chlorine dioxide concentration of about 1 ppm to about 35 ppm.

12. The disinfectant solution of claim 10 wherein the pH of the disinfectant solution is about 2.5.

13. The disinfectant solution of claim 11 wherein the pH of the disinfectant solution is about 2.5.

14. The disinfectant solution of claim 10, wherein the acidic solution includes GRAS listed acids.
